SEQUENCE LISTING (30 sequences)

SEQ ID NO: 1 - protein, 6 aa, Artificial Sequence
  Description of Artificial Sequence Synthetic 6xHis tag
HHHHHH

SEQ ID NO: 2 - protein, 4 aa, Artificial Sequence
  Description of Artificial Sequence Synthetic peptide
VDAL

SEQ ID NO: 3 - protein, 7 aa, Artificial Sequence
  Description of Artificial Sequence Synthetic 7xHis tag
HHHHHHH

SEQ ID NO: 4 - protein, 15 aa, Artificial Sequence
  Description of Artificial Sequence Synthetic peptide
LHHILDAQKMVWNHR

SEQ ID NO: 5 - protein, 4 aa, Artificial Sequence
  Description of Artificial Sequence Synthetic peptide
GSHM

SEQ ID NO: 6 - protein, 8 aa, Artificial Sequence
  Description of Artificial Sequence Synthetic peptide
PLGLDLAR

SEQ ID NO: 7 - DNA, 39 bp, Artificial Sequence
  Description of Artificial Sequence Synthetic primer
cacgtcgacgcattagcggatggaaaaatactactccat

SEQ ID NO: 8 - DNA, 30 bp, Artificial Sequence
  Description of Artificial Sequence Synthetic primer
cacgtcgagaccttgcgcgtcccagcttag

SEQ ID NO: 9 - DNA, 27 bp, Artificial Sequence
  Description of Artificial Sequence Synthetic primer
ttcgagctcgcggatggaaaaatactc

SEQ ID NO: 10 - DNA, 54 bp, Artificial Sequence
  Description of Artificial Sequence Synthetic primer
tccgtcgactacccgtacgacgtcccggactacgcggcattagcggatggaaaa

SEQ ID NO: 11 - DNA, 30 bp, Artificial Sequence
  Description of Artificial Sequence Synthetic primer
tcgggatccggaaaaatactccataatcaa

SEQ ID NO: 12 - DNA, 36 bp, Artificial Sequence
  Description of Artificial Sequence Synthetic primer
tcgggatccgcattagcggatggaaaaatactccat

SEQ ID NO: 13 - DNA, 24 bp, Artificial Sequence
  Description of Artificial Sequence Synthetic primer
attcatatggtgagcaagggcgag

SEQ ID NO: 14 - DNA, 27 bp, Artificial Sequence
  Description of Artificial Sequence Synthetic primer
cacgtcgaccttgtacagctcgtccat

SEQ ID NO: 15 - DNA, 24 bp, Artificial Sequence
  Description of Artificial Sequence Synthetic primer
attcatatgaataagcgagaccta

SEQ ID NO: 16 - DNA, 27 bp, Artificial Sequence
  Description of Artificial Sequence Synthetic primer
tccgtcgacctgaggcatgtagccgcc

SEQ ID NO: 17 - DNA, 30 bp, Artificial Sequence
  Description of Artificial Sequence Synthetic primer
ggccatatgatgaaggataacaccgtgcca

SEQ ID NO: 18 - DNA, 30 bp, Artificial Sequence
  Description of Artificial Sequence Synthetic primer
tccgtcgactttttctgcactacgcaggga

SEQ ID NO: 19 - DNA, 27 bp, Artificial Sequence
  Description of Artificial Sequence Synthetic primer
agccatatgtatgctccagaggccctt

SEQ ID NO: 20 - DNA, 27 bp, Artificial Sequence
  Description of Artificial Sequence Synthetic primer
tccgtcgacagcagggttggggcgtgt

SEQ ID NO: 21 - DNA, 19 bp, Artificial Sequence
  Description of Artificial Sequence Synthetic primer
cttccatatggctcccatg

SEQ ID NO: 22 - DNA, 19 bp, Artificial Sequence
  Description of Artificial Sequence Synthetic primer
taacgtcgacctcgagtcc

SEQ ID NO: 23 - protein, 8 aa, Artificial Sequence
  Description of Artificial Sequence Synthetic peptide
PLGLDLAR

SEQ ID NO: 24 - protein, 8 aa, Artificial Sequence
  Description of Artificial Sequence Synthetic peptide
VDALADGK

SEQ ID NO: 25 - protein, 7 aa, Artificial Sequence
  Description of Artificial Sequence Synthetic peptide
LEHHHHH

SEQ ID NO: 26 - protein, 4 aa, Artificial Sequence
  Description of Artificial Sequence Synthetic peptide
ADGK

SEQ ID NO: 27 - protein, 8 aa, Artificial Sequence
  Description of Artificial Sequence Synthetic peptide
YAALADGK

SEQ ID NO: 28 - protein, 6 aa, Artificial Sequence
  Description of Artificial Sequence Synthetic peptide
ELADGK

SEQ ID NO: 29 - protein, 8 aa, Artificial Sequence
  Description of Artificial Sequence Synthetic peptide
GSALADGK

SEQ ID NO: 30 - protein, 6 aa, Artificial Sequence
  Description of Artificial Sequence Synthetic peptide
LGSIGK

(19) United States
(12) Patent Application Publication
(10) Pub. No.: US 2013/0012687 A1
(43) Pub. Date: Jan. 10, 2013

(54) INDUCIBLE SELF-CLEAVING PROTEASE TAG AND METHOD OF PURIFYING RECOMBINANT PROTEINS USING THE SAME

(75) Inventors: Matthew Bogyo, Palo Alto, CA (US); K. Christopher Garcia, Palo Alto, CA (US); Aimee Shen, Palo Alto, CA (US); Patrick J. Lupardus, Palo Alto, CA (US)

(73) Assignee: The Board of Trustees of the Leland Stanford Junior University

(21) Appl. No.: 13/261,221

(22) PCT Filed: Sep. 21, 2010
(86) PCT No.: PCT/US10/02571

§ 371 Date: Sep. 17, 2012

Related U.S. Application Data
(60) Provisional application No. 61/272,401, filed on Sep. 21, 2009.

(51) Int. Cl. C12P 21/06; C07K 14/00

(52) U.S. Cl. 530/350; 435/68.1

(57) ABSTRACT

A method of purifying a protein is disclosed which entails: a) fusing a site-specific affinity-tagged cysteine protease domain to a target protein to form a tagged fusion protein; b) activating the site-specific cysteine protease domain of the tagged fusion protein by subjecting the site-specific affinity-tagged cysteine protease domain to an inducer, which induces autoprocessing at a cleavage site; thereby releasing untagged target protein; and c) isolating the untagged target protein.

### BACKGROUND OF THE INVENTION

1. Field of the Invention

The present invention provides an inducible, self cleaving protease tag, and a method of purifying recombinant proteins using the same.

2. Description of the Background

The availability of simple, reliable, and cost-effective methods for recombinant protein purification is critical for the work of high throughput structural and proteomic centers and many individual researchers alike. While the addition of affinity tags such as poly-His and glutathione transferase (GST) to target proteins has greatly simplified purification strategies, it is often difficult to obtain soluble recombinant protein. As a result, affinit-tagged target proteins are often additionally fused to small proteins such as NusA and SUMO to improve their solubility, expression, and stability.

However, these tags can alter the biological activity of target proteins and interfere with protein crystallization studies. Therefore many biological and biomedical applications require tag removal from the target protein. Most commonly used methods require the addition of exogenous site-specific proteases to cleave the affinity tag off the target protein at engineered sites. Unfortunately, the need for high levels of endoprotease for extended periods of time can result in unwanted cleavage events within the target protein. Furthermore, these endoproteases are costly, often exhibit poor solubility, an require the inclusion of additional chromatography steps to remove the exogenous protease.

### SUMMARY OF THE INVENTION

Accordingly, the present invention provides an inducible, sell-cleaving or autoprocessing protease tag, which may be used advantageously in the purification of recombinant proteins.

It is also an object of the present invention to provide a method of purifying recombinant proteins using tho inducible, self-cleaving protease tag.

It is, moreover an object of the present invention to provide a method of purifying recombinant proteins, wherein the purification, cleavage and separation of untagged protein from an endoprotease in condensed into a single step.

More particularly, it is an object of the present invention to provide a method of purifying a protein, which entails: a) fusing a site-specific affinity-tagged cysteine protease domain (CPD) protease to a target protein b) activating the site-specific CPD by subjecting the site-specific affinity-tagged CPD to an inducer, which induces autoprocessing at a cleavage site; thereby releasing untagged target protein; and c) isolating the untagged target protein.

### BRIEF DESCRIPTION OF THE DRAWINGS

FIG. 1 illustrates a CPD fusion protein purification system. (a) Schematic of target protein purification using the CPD, described in detail in the text. (b) Schematic of CPD fusion protein. The SalI restriction site, which encodes the P4 and P3 residues Val and Asp, respectively, and the remaining P2-P4′ residues contained within the CPD are shown. Prime positions refer to residues C-terminal to the autocleavage site, which is demarcated as a black vertical line. The composition of the residues added to the C-terminus of target proteins following autoprocessing can vary between one and four residues as described in FIG. 3. Currently, the CPD system functions as a C-terminal fusion to target proteins and thus complements existing methods in which the affinity tag can only be applied as an N-terminal fusion13. (c) Purification of GFP using the CPD-His6 tag. GFP-CPD His6 bound to Ni2+-NTA resin was incubated with increasing amounts of InsP6 for 2 hrs at 4° C. GFP released into the supematant was collected; Ni2+-bound proteins were then eluted from the resin by the addition of 200 mM imidazole. Collected fractions were analyzed by SDS-PAGE. (d) Visual analysis of GFP released into the supernatant fraction upon InsP6 addition to immobilized GFP-CPD-His6 fusion protein.

FIG. 2 illustrates purification of test proteins using the CPD system. SDS-PAGE analysis using Coomassie stain of purifications of (a) biotin ligase (BirA, 35 kDa) using either the CPDHis6 or GST-His6 fusion tags, (b) CAD domain of STIMI (14 kDa) using either the CPDHis6 or GST-His6 fusion tags. Asterisks indicate GST-STIMI(CAD)-His6 derived degradation products, and (c) mouse macrophage metalloelastin (MMP12) using CPDHis6 or His6-affinity tags. The diagonal arrows indicate a His6-tagged truncated MMIP12 product that is also observed during MMP12 purification from inclusion bodies. Large asterisk indicates a putative chaperone protein that co-purifies with MMP12VDAL. In all cases, His6-tagged proteins bound to the Ni2+-NTA resin were incubated with 50 μM InsP6 for 1 hour at room temperature, and the resin was washed three times, followed by elution of Ni2+bound proteins by 200 mM imidaole. CL, cleared lysate, +, IPTG induced culture, FT, flowthrough, IP6, elution from InsP6 incubation, E, imidazole elution prior to InsP6 addition.

FIG. 3 is a schematic of pET-CPD expression vectors.

FIG. 4 illustrates purification of the gp130 intracellular domain (ICD) using hte CPD-His6 tag.

FIG. 5 illustrates purification of PfSENP1 using the CPD system.

FIG. 6 illustrates additional purification of CPD-cleaved MMP12vdal.

### DETAILED DESCRIPTION OF THE PREFERRED EMBODIMENTS

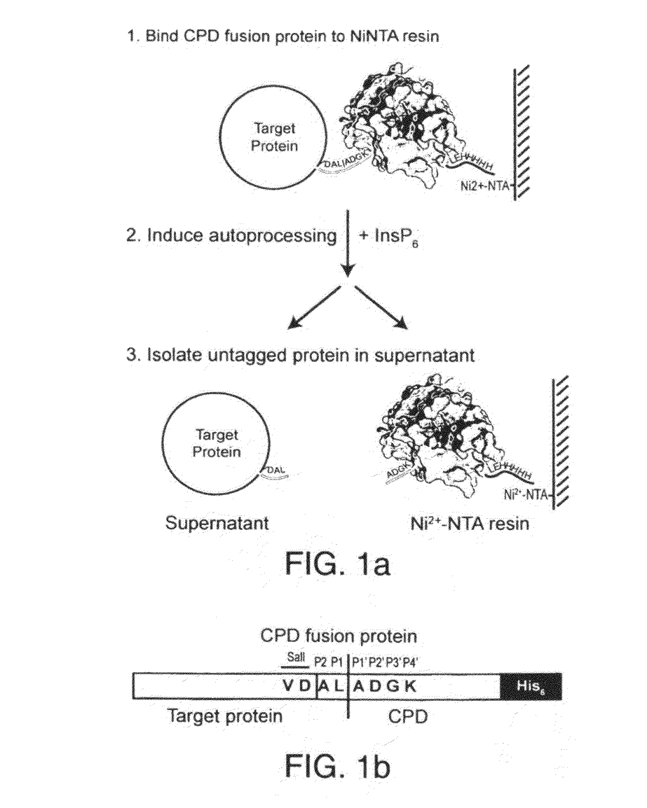

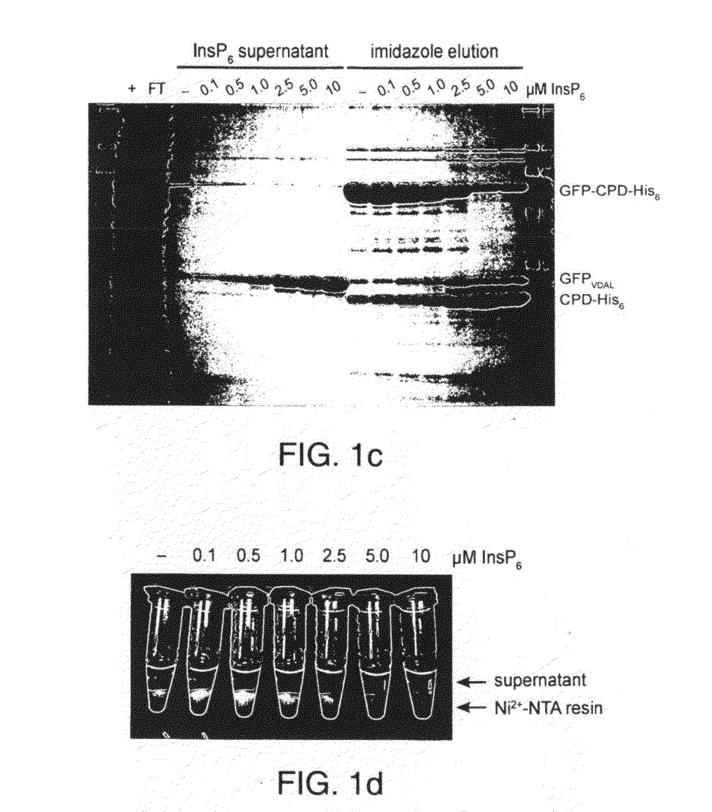

To circumvent the above disadvantages, the present inventors have developed an on-bead cleavage purification system in which a site-specific affinity-tagged protease is fused directly to the target protein (FIGS. 1a and b). A principal advantage of this approach is that affinity purification, cleavage, and separation of the untagged target protein from the endoprotease is condensed into a single step. This system combines the simplicity of onestep purification systems with many of the advantages of affinity tags, such as enhanced expression, integrity, and solubility of target proteins.

An important element of this purification method is the use of the Vibrio cholerae MARTX toxin cysteine protease domain (CPD). The CPD exhibits several properties that make it amenable to its development into an inducible autocleaving protease tag First, the CPD is a highly specific protease that cleaves exclusively after Leu residues4. In the native toxin, the CPD processes the MARTX toxin within interdomain junctions to release discrete effector domains. Secondly, the CPD is selectively activated by the cukaryotic-specific small molecule inositol hexakisphosphate (InsP6). Since InsP6 is absent from bacterial cells6, when the CPD-His6 tag is fused to the C-terminus of target proteins and expressed in E. coli, a CPD-His6 fusion protein can be purified from bacterial lysates in a protease-inactive form using imidzaole affinity chromatography (IMAC, FIG. 1a). Addition of InsP6 to the immobilized fusion protein induces autoprocessing at the P1 Leu cleavage site (with PI referring to the residue N-terminal to the scissile bond), which is located at the target protein-CPD junction. This processing event releases the untagged target protein into the supernatant, while the His6-tagged CPD remains immobilized on the Ni2+- NTA resin.

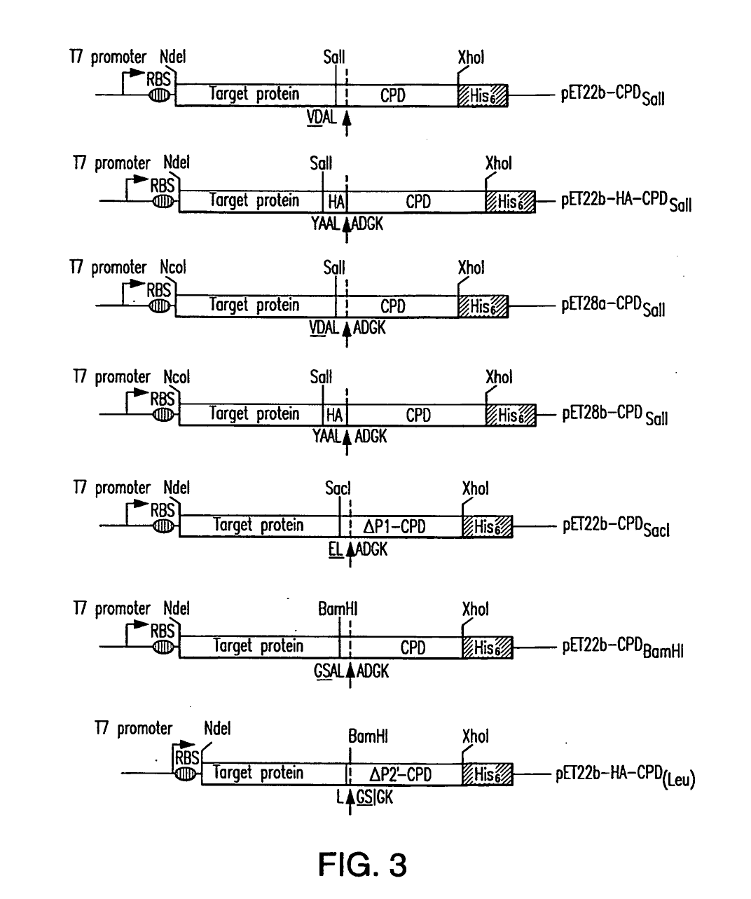

To demonstrate the feasibility of this system, the present inventors constructed pET expression vectors in which DNA encoding the CPD was cloned in to the SalI restriction site to generate pET-CPD vectors (FIG. 3). The fusion protein produced upon iPTG induction of E. coli harboring these vectors carries the P2-P1 residues of the natie CPD (Ala-Leu, respectively) and the P4-P3 residues encoded by the SalI site (Val-Asp, respectively) (FIGS. 1a and b). Upon CPD-mediated autoprocessing of the fusion protein, the untagged target protein released form the resin carries four additional residues (Val-Asp-Als_leu) and the C-terminus. This C-terminal addition can be reduced to two amino acids (Glu-Leu) by cloning into the SacI site, or to a single amino acid (Leo) by cloning into the BamHI site and adding a Len condon to the 3′ cloning primer (FIG. 3).

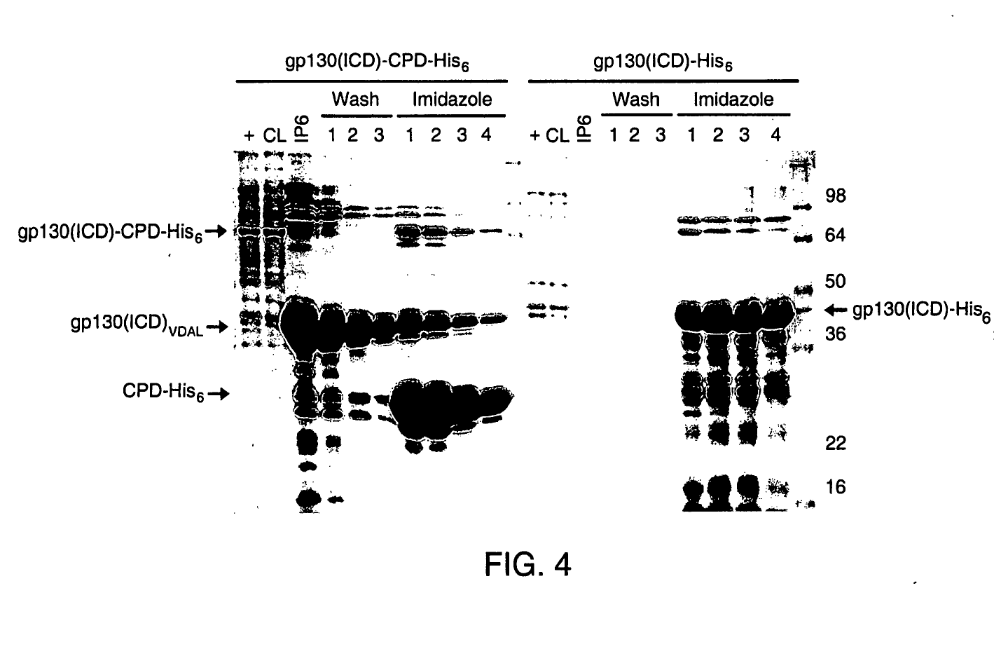

As a proof-of-principle, the present inventors expressed and purified green fluorescent protein (GFP) as a fusion to CPD-His6 using IMAC; addition of increasing amounts of InsP6s stimulated the release of GFP from the Ni2+-NTA agarose beads (bead eluate, FIG. 1c). Since other site-specific proteases that are used to remove fusion tags have been observed to cleave target proteins at secondary sites, we sought to examine whether the CPD would spuriously cleave target proteins. To assess the fidelity of CPD-mediated processing of fusion proteins, we examined whether the CPD would cleave an intrinsically disordered protein after Leu residues within the target protein. We used the intracellular domain (ICD) of the cytokine receptor gp130 as a test substrate, since it is unstructured in solution by NMR7 and contains multiple Leu residues that might serve as cleavage substrates. The ICD-CPD-His6 fusion protein was expressed and purified from E. coli lysates using IMAC, and CPD-mediated cleavage of the immobilized fusion protein was activated by InsP6 addition. Autoprocessing occurred exclusively at the ICDCPD interdomian junction, with a single protein equivalent to the size of His6-tagged ICD being released into the supernatant fraction (FIG. 4). These results are consistent with our previous observation that CPD-mediated transcleavage is highly inefficient and strongly suggest that the CPD will not promiscuously cleave target proteins.

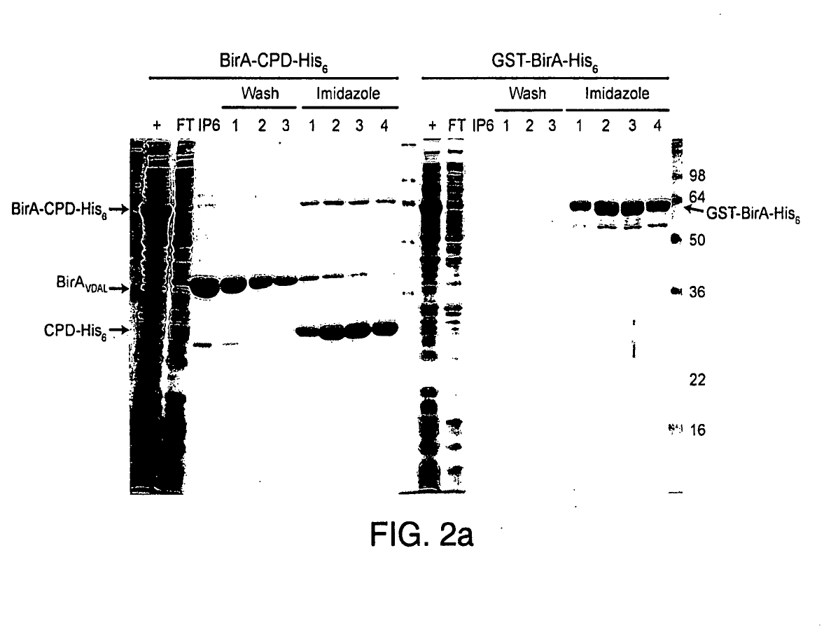

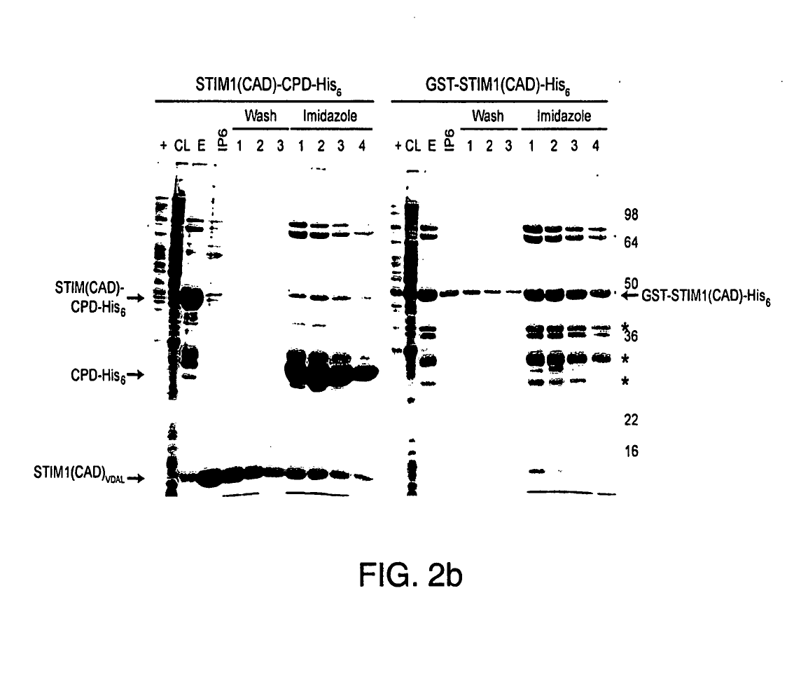

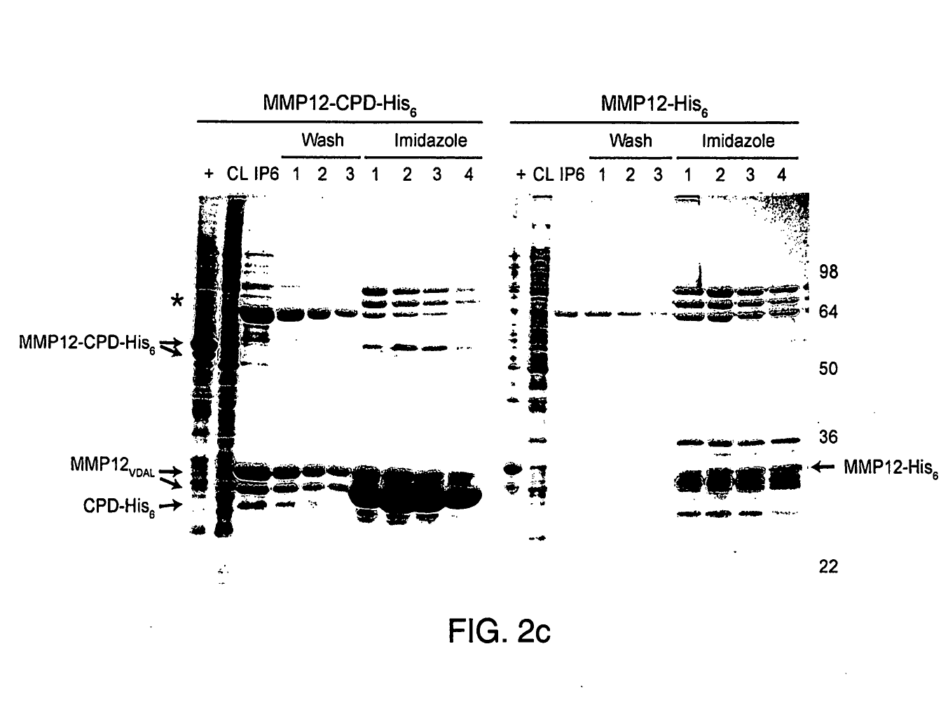

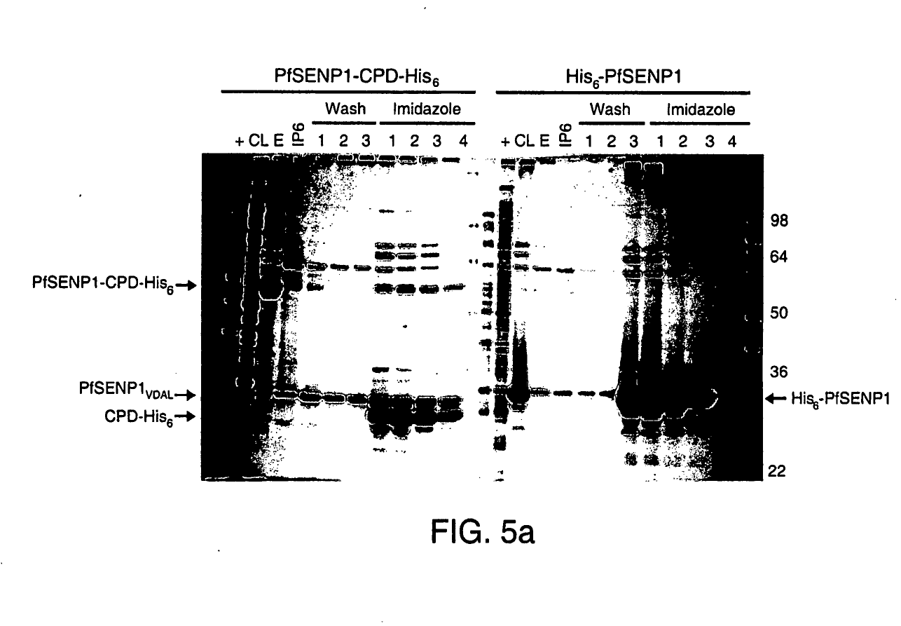

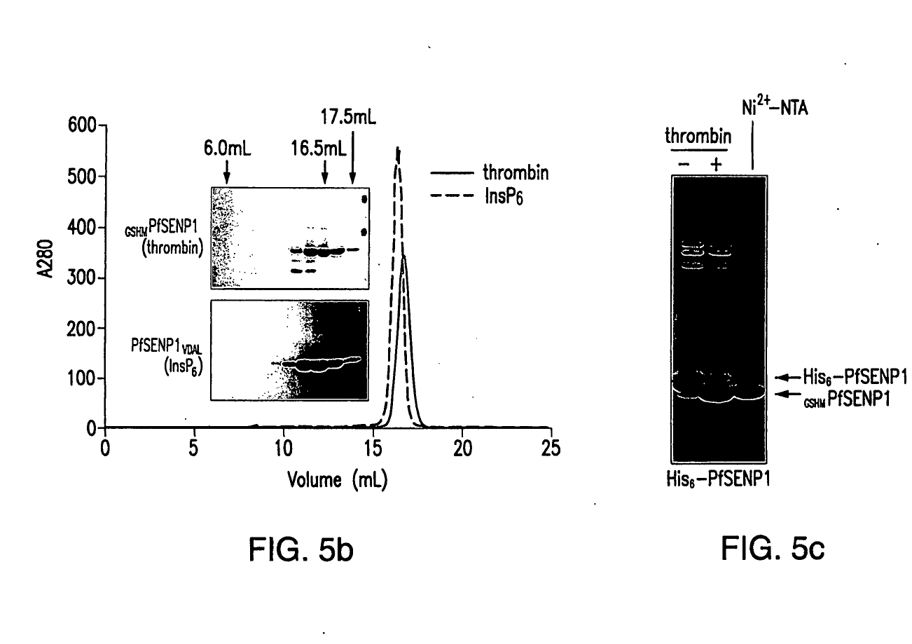

We noticed that the expression of the ICD-CPD-His6 fusion protein was approximately two-fold higher than the ICD-His6 protein in E. coli lysates (FIG. 4). This result suggested that the CPD might generally enhance target protein expression and/or solubility levels. To test this hypothesis, we compared the expression and solubility levels of CPD fusions to several other target proteins carrying either a His6-tag and /or GST-fusion tag (FIG. 2, FIG. 5, Table 1). In all cases, the presence of the CPD-His6 fusion tag increased the expression and solubility of target proteins. For example, fusion of the CPD-His6 tag to biotin ligase(BirA) from E. coli (BirA-CPD-His6_raised BirA expression levels by three-fold over the GST-BirA construct8 (FIG. 2a and Table 1).

The CPD purification system also enhanced the expression, as well as purity, of a previously uncharacterized SUMO/Sentrin-specific peptidase 1 (SENP1) from the parasitic pathogen Plasmodium falciparum, the causative agent of malaria (FIG. 5). Although PfSenP1 carrying an N-terminal His6-tagged can be readily expressed and purified from E. coli, the N-terminal His7-tag must be removed by the addition of thrombin followed by multiple chromatography steps (Table 2). In contrast, when PfSENP1 is expressed as a fusion to CPD-His6 and released as untagged PfSENP1 upon InsP6 addition, only one minor contaminant co-purifies with PfSENP1. This variant is easily removed using gel filtration chromatography, and the untagged PfSENP1 is of sufficient purity that we have used it to obtain diffraction-quality crystals. Although the heterologous expression of P. falciparum proteins in E. coli is typically challenging, we have observed that this system can enhance the expression and purification of other parasite proteins.

In addition to augmenting the expression of target proteins, the CPD-His6 fusions can protect target proteins from proteolytic degradation. This can be demonstrated by fusing the CRAC-activation domain (CAD) of the ER calcium sensor STIMI to the CPD (FIG. 2b). CAD is a a small 107 aa polypeptide that activates Ca2+ release-activated Ca2+ (CRAC) channels by binding to the CRAC channel protein Orai 1. Until now, largescale expression and purification of this important regulatory domain has proven difficult due to its apparent instability even when fused to GST (FIG. 2b). Using the CPD system, we were able to obtain significant quantities of the intact CAD protein, which has subsequently been used in high-throughput screens for Orai1-CAD binding partners.

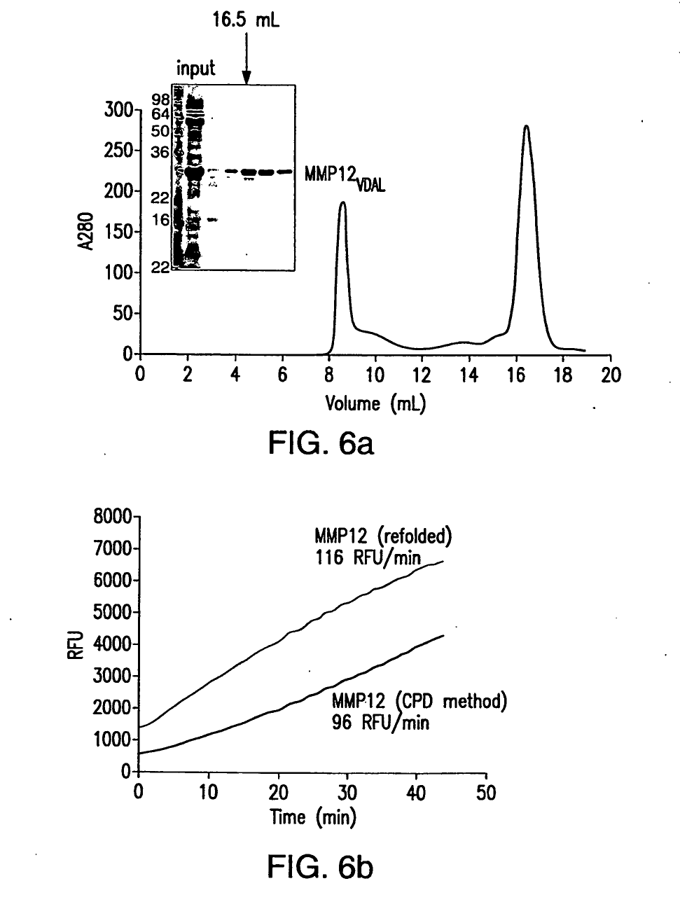

Moreover, the CPD purification system also increased the solubility of difficult-to express proteins. Fusion of the mouse macrophage metalloelastase (MMP12) to CPDHis6 facilitated its purification from the soluble fraction of E. coli lysates, whereas His6-tagged MMP12 remained largely insoluble (FIG. 2c). The currently used method for purification of His6-tagged MMP12 is a laborious procedure that requires solubilization of MMP12 inclusion bodies, refolding over multiple days, followed by anion and cation exchange chromatography. The CPD purification system dramatically simplifies this purification procedure, allowing soluble, active MMP-12 to be isolated in approximately 7 hours (FIG. 6 and Table 3).

Collectively, these results imply that the one-step purification systems such as the intein-chitinbinding-domain (CBD) and sortase-His6. While these systems simplify the purification of well-expressed proteins, the large size of the intein-CBD fusion tag can decrease target protein solubility, and sortase-His6 fusion tags do not increase target protein solubility. Furthermore, unlike self-cleaving elastin-like polypeptide (ELP) tags, fusion proteins do not need to be subjected to the temperature cycles, pH shifts, or high salt concentrations, a feature that is critical for the purification of the intractable proteins. Based on the properties reported here, the CPD could replace the intein-tag in the self-cleaving-ELP system and potentially improve the solubility of ELP-tagged proteins while retaining their self-cleavability. Indeed, a considerable strength of this method is that the CPD remains active over a wide range of conditions. CPD-mediated cleavage is complete within 1-2 hrs at temperatures between 4° C. and 37° C., requires only micromolar of the small molecule InsP6 (an abundant and inexpensive reagent), and occurs efficiently both in the presence of standard protease inhibitor cocktails and in the absence of salt. This latter property carries the additional advantage of allowing the user to determine the buffer system in which to elute the target protein, which eliminates the need for desalting or buffer exchange steps that can reduce protein yields. Thus, the CPD system allows for considerable flexibility in optimizing purification procedures, as is often necessary for uncharacterized target proteins.

This versatility, combined with our observation that it can advantageously improve the solubility and integrity of difficult-to-express proteins (FIG. 2 and FIG. 6), indicates that it will have widespread utility in biological research. The simplicity of this system will also make it amenable for large-scale proteomic, structural genomic, and commercial applications by eliminating the cost and complexity associated with exogenous sitespecific proteases, potentially permitting its use in robotic systems for constructing protein arrays for screening purposes. Furthermore, it is also possible to generate mutants of the CPD that require high concentrations of InsP6 for activation that would be suitable for use in Eukaryotic expression systems.

### TERM DEFINITIONS

“CPD” means cysteine protease domain.

“MARTX” means multifunctional, autoprocessing RTX toxins produced by certain bacteria. “Inducer” means a small molecule that induces autoprocessing at a cleavage site. In the present invention, that induced autocleavage releases an untagged target protein.

In more detail, FIG. 3 is a schematic of pET-CPD expression vectors. Bent arrow, T7 promoter, Oval (RBS), ribosome binding site, green rectangle, target protein, grey rectangle, CPD, V cholera MARTX (aa. 3440-3650), darker gray rectangle, ΔP1-CPD, V. cholera MARTIX (aa. 3442-3650), darkest rectangle, ΔP2′-CPD, V. cholera MARTX (aa. 3444-3650), black rectangle, His6-tag, white rectangle, HA-tag. The dotted vertical line and arrow indicate the CPD cleavage site. Residues added onto the C-terminus of the target protein following CPD-mediated cleavage, and the relevant restriction site are shown. The composition of the amino acids added to the C-terminus of the target protein can be varied depending on the cloning site and pET-CPD vector used. It should be noted that the P1 Leu shown for pET22b-CPDBamHI-Leu must be encoded in the 3′ cloning primer of the target gene (i.e. supplied by the target gene). Since the primary substrate specificity determinant for the CPD is a P1 Leu, and P2 and P3 residues are not strongly recognized by the CPD substrate binding pocket, the fusion protein produced from pET22b-CPDBamHI-LEU is efficiently autoprocessed by the CPD. Both pET22b and pET28a vector backbones were used to construct the CPD expression vectors.

FIG. 4 illustrates purification of the gp130 intracellular domain (ICD) using the CPDHis6 tag. Gp130 ICD(CPD)-His6 or gp130(ICD)-His6 bound to Ni2+-NTA resin was incubated with 100 μM InsP6 for 2 hr at room temperature; the resin was washed four times, followed by elution of Ni2+-bound proteins by 200 mM imidazole. Purification fractions were analyzed by SDS-PAGE followed by Coomassie staining. CL, cleared lysate, FT, flowthrough, IP6, elution from InsP6 incubation.

FIG. 5 illustrates purification of PISENP1 using the CPD system. (a)SDS-PAGE analysis using Coomassie staining of Plasmodium falcipaurm SENPI (PfSENP I, 25 kDa)purification using either the CPD-His6 or His6-affinity tags. PfSENP1-CPD-His6 or His6-PfSENP1 bound to the Ni2+-NTA resin was incubated with 100 μM InsP6 for 2 hr at room temperature: the resin washed three times, and wash fractions were collected. Ni2+-bound proteins were eluted by adding 200 mM imidazole, +, IPTG induced culture, CL, cleared lysate, E, imidazole elution prior to InsP6 addition, IP6, elution from InsP6, (b) UV trace PfSENP1 further purified by gel filtration chromatography following His6-tag removal. Inset, Coomassie staining of gel filtration fractions of PfSENP1 purifications. Thrombin refers to PfSENP1 purified thrombin-mediated removal of the N-terminal His6-tag, while InsP6 refers to InsP6-induced, CPD-mediated removal of the C-terminal CPD-His6-tag. The residues added to the resulting PfSENP1 protein are indicated; N-terminal GSHM for PfSENP1 (thrombin cleavage) and C-terminal VDAL for PfSENP1 (InsP6-activated CPD cleavage). (c) Coomassie staining of fractions taken during His6-PfSENP1 purification prior to thrombin incubation (−), following 12 hr thrombin incubation (+), and following subtractive IMAC to remove uncleaved His6-PfSENP1 (Ni2+-NTA). The yield of PfSENP1 diminished with each experimental manipulation.

FIG. 6 illustrates additional purification of CPD-cleaved MMP12VDAL. (a) Purification of MMP12VDAL by gel filtration chromatography. Inset, Coomassie staining of SDS-PAGE analysis of gel filtration fractions of MMP12VDAL. (b) MMP12 Flourogenic substrate assay. The activity of MMP12 purified under denaturing conditions and refolded (MMP12 (Refolded)) and MMP12VDAL purified using the CPD system against a standard flourogenic substrate were compared. Comparable rates of flourogenic substrate cleavage are observed for MMP12 purified by the CPD method relative to the refolding method.

The results obtained and observed are summarized in the tables below.

### Tables

TABLE 1
Target proteins expressed and purified by CPD purification method

| | Yield^{a} | Yield | |
|---|---|---|---|
| | (mg/L | (nmol/L | |
| Target protein | culture) | culture) | Activity |
| GFP_{VDAL} (CPD | 3.3 | 105 | Fluorescence at 511 nm |
| method) | | | |
| Gp130(ICD)_{VDAL} | 5.9 | 188 | n/a |
| (CPD method) | | | |
| Gp130(ICD)-His₆ | 3.7 | 115 | n/a |
| BirA_{VDAL} (CPD | 10.9 | 202 | Biotinylates |
| method) | | | LHHILDAQKMVWNHR |
| | | | BirA biotinylation site |
| GST-BirA-His₆ | 12.0 | 90 | Biotinylates |
| | | | LHHILDAQKMVWNHR |
| | | | BirA biotinylation site |
| PfSENP1_{VDAL} (CPD | 2.0 | 67 | Cleaves PfSUMO |
| method) | | | |
| _{GSHM}PfSENP1 | 1.4 | 46 | Cleaves PfSUMO |
| STIM1(CAD)_{VDAL} | 2.1 | 148 | Binds Orail |
| GST-STIM1(CAD)- | 2.5^{b} | 62 | n/a |
| His₆ | | | |
| MMP12_{VDAL} (CPD | 1.4 | 47 | Cleaves flourogenic peptide |
| method) | | | substrate |
| | | | Mca-PLGLDL(Dpa)AR |
| MMP12 (refold) | 23 | 767 | Cleaves flourogenic peptide |
| | | | substrate |
| | | | Mca-PLGLDL(Dpa)AR |

^{a}Protein yield per litre of culture
^{b}Yield difficult to assess since GST-fusion protein degrades and falls out of solution over time

TABLE 2
Comparison of CPD-mediated purification of untagged
PfSENP1 to thrombin-mediated purification

| | PfSENP1-CPD-His₆ | His₆-PfSENP1 |
|---|---|---|
| Step 1 | Prepare soluble lysate (1 hr) | Prepare soluble lysate (1 hr) |
| Step 2 | IMAC purification (2 hr) | IMAC purification (2 hr) |
| Step 3 | On-bead cleavage; | Imidazole elution |
| | collect supernatant (2 hr) | |
| Step 4 | Concentrate protein (0.5 hr) | Buffer exchange and concentrate |
| | | protein (0.5 hr) |
| Step 5 | Gel filtration | Thrombin cleavage overnight |
| | chromatography (1 hr) | (>12 hr) |
| Step 6 | Concentrate protein (0.5 hr) | Remove His₆-tag and uncleaved |
| | | fusion with IMAC (1 hr) |
| Step 7 | | Concentrate protein an buffer |
| | | exchange (0.5 hr) |
| Step 8 | | Gel filtration chromatography |
| | | (1 hr) |
| | | Concentrate protein (0.5 hr) |
| Total time | 5 hr | >17.5 hr |

TABLE 3
Comparison of CPD method to published method for
purifying matrix metalloelastin (MMP12)

| | MMP12-CPD-His₆ | MMP12-His₆¹ |
|---|---|---|
| Step 1 | Prepare soluble lysate (1 hr) | Prepare and dissolve inclusion |
| | | bodies (16 hr) |
| Step 2 | IMAC purification (2 hr) | Protein refolding by dialysis in |
| | | 1/20 volume (48 hr) |
| | | a. 24 hr - 6M Urea, 4 L |
| | | b. 24 hr - 3M Urea, 4 L |
| | | c. 24 hr - 1M Urea |
| Step 3 | On-bead cleavage; | Load partially refolded protein on |
| | collect supernatant (2 hr) | anion and cation exchange tandem |
| | | columns (2.5 hr) |
| Step 4 | Concentrate protein (0.5 hr) | Wash columns with three buffers |
| | | to fully refold protein on column |
| | | (2 hr) |
| Step 5 | Gel filtration | Elute protein from cation exchange |
| | chromatography (1 hr) | column (0.5 hr) |
| Step 6 | Concentrate protein (0.5 hr) | Concentrate protein (0.5 hr) |
| Total | 7 hr | 3-4 days |
| time | | |

TABLE 4
Primers used in examples.

| # | Name | Sequence^{a} | RE^{b} |
|---|---|---|---|
| 1 | 5′ Sall A50CPD | CACGTCGACGCATTAGCGGATGGAAAAATACTACTCCAT | Sall |
| 2 | 3′ Xhol CPD | CACGTCGAGACCTTGCGCGTCCCAGCTTAG | Xhol |
| 3 | 5′ Scal CPD | TTCGAGCTCGCGGATGGAAAAATACTC | Scal |
| 4 | 5′ Sall HA-CPD | TCCGTCGACTACCCGTACGACGTCCCGGACTACGCGGCATTAGCGGATGGAAAA | Sall |
| 5 | 5′ BamHl CPD_{Leu} | TCGGGATCCGGAAAAATACTCCATAATCAA | BamHl |
| 6 | 5′ BamHl CPD | TCGGGATCCGCATTAGCGGATGGAAAAATACTCCAT | HamHl |
| 7 | 5′ Ndel gfp | ATTCATATGGTGAGCAAGGGCGAG | Ndel |
| 8 | 3′ Sall gfp | CACGTCGACCTTGTACAGCTCGTCCAT | Sall |
| 9 | 5′ Ndel gp130(1CD) | ATTCATATGAATAAGCGAGACCTA | Ndel |
| 10 | 3′ Sall gp130(1CD) | TCCGTCGACCTGAGGCATGTAGCCGCC | Sall |
| 11 | 5′ Ndel BirA | GGCCATATGATGAAGGATAACACCGTGCCA | Ndel |
| 12 | 3′ Sell BirA | TCCGTCGACTTTTTCTGCACTACGCAGGGA | Sall |
| 13 | 5′ Ndel CAD | AGCCATATGTATGCTCCAGAGGCCCTT | Ndel |
| 14 | 3′ Sall CAD | TCCGTCGACAGCAGGGTTGGGGCGTGT | Sall |
| 15 | 5′ Ndel MMP12 | CTTCCATATGGCTCCCATG | Ndel |
| 16 | 3′ Sall MMP12 | TAACGTCGACCTCGAGTCC | Sall |

^{a}Restriction enzyme sequences are underlines, and HA tag is shown in italics. ^{b}Restriction site

TABLE 5
Strains used in examples

| Strain | Genotype and relevant features | Reference |
|---|---|---|
| 41 | BL21(DE3) | Novagen |
| 7 | DH5α | D. E. Cameron |
| 269 | pET22b in DH5α | D. E. Higgins |
| 195 | pET28a in DH5α | E. Ponder |
| 330 | pET22b-CPD_{salI} | Present invention |
| 329 | pET28a-CPD_{salI} | Present invention |
| 331 | pET22b-HA-CPD_{salI} | Present invention |
| 373 | pET28a-HA-CPD_{salI} | Present invention |
| 374 | pET22b-CPD_{BamHI-Leu} | Present invention |
| 375 | pET22b-CPD_{BamHI} | Present invention |
| 197 | pET22b-GFP-CPD | Present invention |
| 371 | pET22b-gp130(ICD)-CPD | Present invention |
| 372 | pET21a-gp130(ICD) | 2 |
| 228 | pET22b-BirA-CPD | Present invention |
| 183 | pGEX4T1-BirA | P. J. Lupardus |
| 360 | pET22b-PfSENP1-CPD | 3 |
| 361 | pET28a-PfSENP1 | 3 |
| 324 | pET22b-STIM1(CAD)-CPD | Present invention |
| 327 | pGEX6-CAD128 | 4 |
| 359 | pET22b-mMMP12 | Present invention |
| 358 | pET41a-mMMP12 | C. Overall |

### Methods

Bacterial growth conditions Overnight bacterial strains were grown at 37° C. in Luria-Bertrani (LB) broth. Antibiotics were used at 100 μg/mL carbenicillin for pET22b vectors expressed in E. coli.

Strain construction Primers used ate listed in Table 4; strains constructed are listed in Table 5. For construction of pET-CPDSalI vectors, DNA encoding Vibrio cholera MARTX toxin amino acids 3440-3650 from Vibrio cholerae N16961 was PCR amplified from genomic DNA using primers #1 and #2. The resulting PCR fragment was cloned into the SalI and XhoI sites of the pET22b and pET28a expression vectors, respectively (Novagen). For construction of the pET-CPDSacI vector, DNA encoding Vibrio cholerae MARTX toxin amino acids 3442-3650 from Vibrio cholerae N16961 was PCR amplified from genomic DNA using primers #3 and #2, and the resulting PCR fragment was cloned into the SacI and XhoI sites of pET22b. To construct the pET-HA-CPDSalI vectors, DNA encoding the HA epitope tag was added to the 5′ end of primer #4, and PCR amplification using primers #4 and #2 was used to fuse the HA tag directly to amino acid 3440 of V. cholera MARTX CPD. The resulting PCR fragment was cloned into the SalI and XhoI sites of the pET22b and pET28a expression vectors, respectively. For construction of the pET-CPDBamHI-Leu vector, DNA encoding Vibrio cholerae MARTX toxin amino acids 3440-3650 from Vibrio cholerae N16961 was PCR amplified from genomic DNA using primers #5 and #2, and the resulting PCR fragment was cloned into BamHI and XhoI sites of pET22b. For construction of the pETCPDBamHI vector, DNA Vibrio cholerae MARTX toxin amino acids 3440-3650 from Vibrio cholerae N 16961 was PCR amplified from genomic DNA using primers #6 and #2 were used, and the resulting PCR fragment was cloned into the BamHI and XhoI sites of pET22b. The pET22b-GFP-CPD construct was cloned by PCR amplifying GFP from pEGFPN3(Clontech) using primers #7 and #8. To construct the pET22b-gp130(ICD)-CPD vector, amino acid 642-918 of gp130 corresponding to the intracellular domain were PCR amplified using primers #9 and #10 and pET21a-gp130(ICD) as a template. The pET22b-BirA-CPD vector was constructed by PCR amplifying the birA gene from the pGEX4T1-BirA template using primers #9 and #10. The pET22b-STIMI(CAD)-CPD plasmid was constructed by PCR amplifying DNA encoding amino acids 342-369 of STIMI using pGEX6-CAD128 as a template and primers #13 and 14. The pET22b-nMMP12-CPD construct was constructed by PCR amplifying the catalytic domain of mouse MMP12 (aa 29-267) using pET41a-mMMP12 as a template using primers #15 and #16. In all cases, the resulting PCR products were cloned into the NdeI and SalI sites of pET22b-CPDSalI.

Protein expression and purification. For purification of His6-tagged CPU fusion proteins, overnight cultures of the appropriate strain were diluted 1:500 into 1 L 2YT media and grown shaking at 37° C. When an OD600 of 0.6 was reached. IPTG was added to 250 μM, and cultures were grown for 3-4 hrs at 30° C. Cultures were pellatized, resuspended in 25 mL lysis buffer (500 nM NaCl, 50 mM Tris-HCL, pH 7.5, 15 nM imidazole, 10% glycerol) and flash frozen in liquid nitrogen. Lysates were thawed, then lysed by sonication and cleared by centrifugation at 15,000×g for 30 minutes. His6-tagged CPD fusion proteins were affinity purified by incubating the lysates in batch with 0.5-1.0 mL Ni-NTA Agarose beads (Qiagen) with shaking for 2-3 hrs at 4° C. the binding reaction was pelleted at 1,500×g, the supernatant was aside, and the pelleted Ni2+-NTA agarose beads were washed three times with lysis buffer. In some cases, 10% of the Ni2+-NTA beads containing immobilized CPD-His6 fusion proteins were removed, pelleted and then His6-tagged eluted using high imidazole buffer (500 nM NaCl, 50 mM Tris-HCL, pH 7.5, 175 nM imidazole, 10% glycerol).

To liberate untagged target proteins into the supernatant fractions, 300-500 μL lysis buffer was added to the Ni2+-NTA beads containing CPD-His6 fusion proteins and the indicated amount of inositol hexakisphosphate (InsP6, Calbiochem) was added. In general, on-bead cleavage was allowed to proceed by nutating the beads in the presence of 50-100 μM InsP6 for 1-2 hr at room temperature or 4° C. The beads were pelleted at 1,500×g, and the supernatant fraction was removed. The beads were then washed 3-4 times with 300-500 μL lysis buffer, and supernatant fractions retained. His6-tagged proteins remaining on the beads (i.e. cleaved CPD-His6) were eluted using high imidazole buffer (500 nM NaCl, 50 mM Tris-HCL, pH 7.5, 175 nM imidazole, 10% glycerol) in 300-500 μL volumes. The elute was repeated 3-4 times, and elute fractions were collected. Purification of His6-tagged proteins lacking the CPD was performed in parallel. This general procedure was followed with the following exceptions: for purification of MMP12 constructs, the cultures were grown at 16° C. overnight after IPTG induction, and 1 mM tris(2-carboxyethyl)phosphine (TCEP) was added to the lysis buffer to prevent misfolding of the protein. PfSENP1 and BirA protein purifications were performed exclusively at room temperature, since at 4° C., protein aggregation was observed. For removal of the His6-tag from His6-PfSENPI, thrombin beads (Calbiochem) that had been washed in PBS were added to the elute His6-PfSENP1, which had been buffer exchanged into PBS. Thrombin cleavage was allowed to proceed with shaking overnight for 12 hr at room temperature. Aliquots were taken before and after thrombin addition to monitor cleavage efficiency. Thrombin cleaved, untagged PfSENP1 was enriched by performing a subtractive Ni2+-NTA pull-down. Untagged PfSENP1 from both methods was then buffer-exchanged into gel filtration buffer (50 mM NaCl, 20 mM Iris pH 8.0). Protein purifications were analyzed by SDS-PAGE and Coomassie staining using GelCode Blue (Pierce). Purified protein concentrations of purified were determined by Bradford assay (Pierce).

Purification of MMP12-His6. MMP12-His6 was purified as previously described with the following modifications. The cell pellet was resuspended in 100 mM NaCl, 100 mM Tris pH 8.0, 5.0 mM EDTA, 0.5 mM DTT, 100 μg/mL lysozyme and stirred for 2 hr. The cells were sonicated then centrifuged at 10,000 rpm for 10 min. The resulting inclusion bodies were washed two times and then resuspended in 50 mL 6M guanidine hydrochloride, 10 mM Iris pH 8.0 by stirring at 4° C. overnight. The mixture was centrifuged at 15,000 rpm for 30 min, and 2 mL aliquots of supernatant were prepared. The supernatant was diluted 1:100 into denaturing buffer (6M Urea, 50 mM Tris pH 8.0, 10 mM CaCl2, 30 mM NaCl, 5 mM DTT) to a final concentration of 0.1-0.2 mg/mL. The protein was then dialyzed for 24 hr in 2 L refolding buffer 1 (3 M Urea, 50 mM Tris pH 8.0, 10 mM CaCl2, 30 mM NaCl, 5 mM DTT). The partially refolded protein was then dialyzed in 4 L of refolding buffer 2 (1 M Urea, 50 mM HEPES pH 7.4, 10 mM CaCl2, 5 mM DTT). The buffer exchanged protein was then purified using tandem 5 mL MonoQ and SP Sepharose (GE Healthcare) at 4° C. After loading the protein on the column, the column was washed with 50 mL of refolding buffer 2 without DTT at 1 M, 0.5, and 0 Murea, respectively. The protein was eluted from the SP column in 500 mM NaCl, 50 mM HEPES ph 7.4, 10 mM CaCl2.

Gel filtration chromatography. Untagged PfSENP1 obtained from either thrombin or InsP6-mediated cleavage was concentrated using 10 kDa Centricon concentrator (Millipore) and buffer exchanged into 50 mM NaCl. 20 mM Tris pH 8.0 and purified on a Superdex 200 10/30 column (GE Healthcare) equilibriated in the same buffer. For MMP12, the gel filtration buffer contained 150 mM NaCl, 50 Mm Tris pH 7.4, 10 Mm TCEP. Gel filtrations were performed at 4° C.

Activity assays. Flourescence of purified GNP at 511 nm was verified using a Molecular Devices fmax plate reader in black 96-well plates and 488 nm excitation. The activity of MMP12 was determined using the flourogenic substrate Mea-PLGLDL(Dpa)AR (Mea, (7-methoxycoumarin-4-yl)acetyl, Dpa, N-3-(2,4-dinitrophenyl)-L-2,3-diaminopropionyl, Anaspec). Reactions were preformed in the assay buffer (50 Mm Tris pH 7, 150 mM NaCl, 10 Mm CaCl2, 0.02% NaN3, 5 mM TCEP) at 37° C. The substrate was used at 10 μM and the protein at 0.2 μM. The substrate hydrolysis was monitored continuously in a fluorescent plate reader (Molecular Devices) using an excitation wavelength of 325 nm and an emission wavelength of 395 mn.

In addition to the embodiments described above, other variations thereof may also be used on accordance with the present invention without departing from the spirit and scope thereof.

For example, aside from the Vibrio cholera MARTX cysteine protease domain (CPD), related MARTX CPDs from Pholorhabdus luminescens and Vibrio vulnificus also autoprocess in the presence of InsP6. Further, Clostridium toxin CPDs also work in the system.

Generally, MARTX toxins produced by Vibrio sp. and Clostridium sp. may be used. Exemplary species of Vibrio are V. anguillarum, V. splendidus and V. vulnificus in addition to V. cholera. Further, these toxins arc specifically described as CPDs. For example, the Clostridium toxins are CPDs derived from the large glucosylating toxins produced by lostridium sp. All of these bacterial species are commercially or readily available to the artisan.

Further, in addition to InsP6 (inositol hexakisphosphate), inositol pentakisphosphate (InsP5) may also be used but generally higher concentrations of the later are required to induce autocleavage.

Moreover, while E. coli is used as a well-known expression system the present invention, other bacterial hosts may also be used to produce the target recombinant protein. For example, Bacillus systems and Lactobacillus lactis systems may be used. Generally, any bacterial host system may be used provided that the expressed target protein is secreted into the media, and the media does not contain either InsP6 or InsP5, for example. Further, it is also possible to use the system in eukaryotic cells when the CPD is mutated to be less responsive to InsP6 and InsP5, for example. Basic cage mutations described in recent literature require higher than physiological concentrations of InsP6 (cystolic concentrations have been reported to be between 5-100 micromolar InsP6) to become activated.

Generally, the present method is conducted at a pH range of about 6.5 to 9.5. However, it is preferred that a pH range of about 7.5 to 8.5 be used. Further, the MARTX CPDs used in the present invention are preferably insensitive, i.e., no loss of protease activity, to salt concentration. For example, the MARTX CPDs used in the present invention generally exhibit little or no loss of activity in the presence of NaCl in a concentration of from 0 to 500 mM.

As noted above, the present method generally affords an increased expression of target proteins. In general, although the extent of increased expression varies from protein to protein, at least a two-fold increase in expression is commonly observed. Yet, greater increases are also observed. See the discussion above regarding a comparison of the expression level for (BirA-CPD-His6) in E. coli and (GST-BirA).

Additionally, various affinity tags may be used in accordance with the present invention being fused to the CPD. For example, steptavidin binding tags (SBP or Nanotag), CBP (Calmodulin binding tag) and Protein C-epitope tag may be mentioned. Further, it is also acceptable to combine the use of affinity-tagged CPD with other fusion or affinity tags. For example, the present inventors have constructed vectors in which the protein of interest can itself carry an affinity tag in addition to the affinity-tagged CPD (like the HA tag depicted in FIG. 3). Dually tagged proteins allow for tandem affinity purification of the target protein.

Finally, the CPD used may be modified to function as an N-terminal fusion. For example, the present inventors have observed that the CPD can cleave itself off a streptavidin support when a C-terminal biotinylated peptide sequence is fused to the CPD.

Having described the above embodiments, it will remain clear that various other changes and modifications may be made without departing from the spirit and scope of the present invention.

1. A method of purifying a protein, which comprises the steps of:
a) fusing a site-specific affinity-tagged cysteine protease domain to a target protein to form a tagged fusion protein;
b) activating the site-specific cysteine protease domain of the tagged fusion protein by subjecting the site-specific affinity-tagged cysteine protease domain to an inducer, which induces autoprocessing at a cleavage site; thereby releasing untagged target protein; and
c) isolating the untagged target protein.

2. The method of claim 1, wherein the cysteine protease domain is a Vibrio sp MARTX cysteine protease domain.

3. The method of claim 1, wherein the inducer is inositol hexakisphosphate, InsP₆.

4. The method of claim 1, wherein the cysteine protease domain is MARTX CPD of Photorabdus luminescense.

5. The method of claim 1, wherein the target protein is expressed
in E. coli.

6. The method of claim 1, wherein the target protein is expressed
in Bacillus.

7. The method of claim 1, wherein the target protein is expressed
in Lactobacillus lactis.

8. The method of claim 1, wherein the target protein is expressed in a eukaryotic cell.

9. The method of claim 1, wherein said isolating comprises separating the target protein from bacterial lysates by affinity chromatography.

10. The method of claim 9, wherein the affinity chromatography is imidazole addinity chromatography.

11. The method of claim 1, wherein the site-specific affinity-tagged CPD is immobilized.

12. The method claim 11, wherein the site-specific affinity-tagged CPD is immobilized on Ni⁺²—NTA resin.

13. The method of claim 1, wherein the site-specific affinity-tagged CPD functions by C- or N-terminal fusion at the target protein.

14. The method of claim 1, wherein the site-specific affinity-tagged CPD enhances expression of the target protein

15. The method of claim 1, wherein site-specific affinity-tagged CPD increases solubility of the target protein.

16. The method of claim 1, wherein the target protein is a parasite protein.

17. The method of claim of claim 16, wherein the parasite is P. falciparum.

18. The method of claim 1, wherein the site-specific affinity-tagged CPD protects the target protein from proteolytic degradation.

19. The method of claim 1, wherein the target protein is MMP12.

20. The method of claim 1, which does not require temperature cycles.

21. The method of claim 1, which is conducted at a pH of 6.5 to 9.5.

22. The method of claim 1, wherein the cysteine protease domain is insensitive to salt.

23. The method of claim 1, which is effected at a temperature of between 4-27° C.

24. The method of claim 1, which is effected in about 1-2 hours.

25. The method of claim 1, wherein the untagged target protein is isolated from supernatant.

26. The method of claim 1, wherein the protein purified is a recombinantly expressed protein.

27. The method of claim 1, wherein the inducer is inositol pentakisphosphate, InsPₛ.

28. A recombinant and purified protein produced by the method of claim 1.
